(19) United States
(12) Patent Application Publication
(10) Pub. No.: US 2004/0138544 A1
(43) Pub. Date: Jul. 15, 2004

(54) Body fluid trap anlyte sensor

(76) Inventors: W. Kenneth Ward, Portland, OR (US); Richard G. Sass, Portland, OR (US); Mark Neinast, Gastonia, NC (US); Ellen Anderson, Tualatin, OR (US); Lawrence B. Jansen, Portland, OR (US)

Correspondence Address: TIMOTHY E SIEGEL, 1868 KNAPPS ALLEY, SUITE 206, WEST LINN, OR, 97068, US

(21) Appl. No.: 10/375,891

(22) Filed: Feb. 27, 2003

Related U.S. Application Data
(63) Continuation-in-part of application No. 10/342,144, filed on Jan. 13, 2003.

(51) Int. Cl. A61B 5/05

(52) U.S. Cl. 600/348

(57) ABSTRACT

A rapid response, single use analyte sensor, adapted to measure an analyte concentration in body fluid. The sensor includes a lancet and a body fluid trapping structure, adapted to trap body fluid on the skin. An electrochemical analyte measurement system measures the analyte concentration in the body fluid that is trapped on the surface of the skin, by the body fluid trapping structure. In one preferred embodiment, the body fluid trapping structure defines an interior volume of only 30 nanoliters of body fluid.

### RELATED APPLICATIONS

This application is a continuation in part of utility patent application Ser. No. 10/342,144 filed on Jan. 13, 2003.

### BACKGROUND OF THE INVENTION

Among the other unpleasant aspects of having the disorder diabetes mellitus is the need to frequently test one's blood glucose concentration. With current technology a diabetic patient must prick his own fingertip or other body part with a lancet in order to withdraw blood from the wound. The fingertip is preferred because of the great number of capillaries located there.

The broach created through the skin by the lancet must be wide enough to permit blood to flow through. Human epidermis around the fingertips is on the order of 1-3 millimeters thick. Also, similar to other flexible, sheet like materials, skin tends to close up on itself if broached. Accordingly the lancet used must create a broach that is wide enough to not be closed by the natural action of the skin.

Moreover, the task of sampling one's own blood has generally required that a flat surface be present for the patient to arrange various test articles including a test strip, a lancet and a cotton ball with alcohol, for sterilizing the wound. As a result, it has heretofore been impossible for a diabetic patient to measure his blood glucose level in a public place without drawing attention to himself. Interviews with diabetic patients indicate that the workplace, where there is frequently a definite lack of privacy and where maintaining the secrecy of personal information may be greatly desired, presents particular difficulties.

A number of disclosures are aimed at easing this requirement by providing an integrated unit having a number of lancets and associated test articles (such as a test strip or a sensing cavity to be filled with blood drawn out from the body) and in which both lancet and test article are contemporaneously moved into test position. These devices tend to use chemical test strips, rendering them rather bulky and typically requiring the user to place a test strip in place before use.

In addition, a number of disclosures are directed at an implantable or insertable sensor, for continuous glucose monitoring. Although this technology appears to bear promise it is desirable to have additional options for the diabetic patient. For example, a method of quickly and easily making an occasional determination of blood glucose concentration would be helpful for patients not wishing to wear a glucose monitor.

Ease of use is not only an important consideration from the perspective of patient comfort, but also from the perspective of patient health. The easier it is for a patient to take his blood glucose level reading, the more frequently he is likely to do so. In turn, with more frequent measurements, the patient is likely to do a better job at regulating his glucose level and thereby avoiding chronic complications in which body tissue is damaged by toxic glucose levels or acute complications in which the patient is in danger of entering a state of hypoglycemic shock. Moreover, by more frequently measuring his or her glucose levels, the patient will likely form a better understanding of his body's response to the consumption of varying types of food and of varying degrees of physical exertion. The better the patient understands his body's response characteristics the better he will be able to tailor his eating, exercise and insulin injection or ingestion regime.

### SUMMARY

In a first separate aspect, the present invention is a rapid response, single use analyte sensor, adapted to measure an analyte concentration in body fluid. The sensor includes a lancet and a body fluid trapping structure, adapted to trap body fluid on the skin. An electrochemical analyte measurement system measures the analyte concentration in the body fluid that is trapped on the surface of the skin, by the body fluid trapping structure.

In a second separate aspect, the present invention is a method of measuring the concentration of an analyte in the body fluid of a patient. The method includes using a rapid response, single use analyte sensor having a lancet, a body fluid trapping structure for trapping body fluid on the skin surface and an electrochemical analyte measurement system adapted to measure an analyte concentration in body fluid that is trapped on the skin surface, by the body fluid trapping structure. In the method, the skin is punctured with the lancet, permitting body fluid to flow into the body fluid trapping structure. The electrochemical analyte measurement system measures the analyte concentration in the body fluid.

The foregoing and other objectives, features and advantages of the invention will be more readily understood upon consideration of the following detailed description of the invention, taken in conjunction with the accompanying drawings.

### BRIEF DESCRIPTION OF THE DRAWINGS

FIG. 1 is a partially schematic representation, partially cross-sectional view of a glucose sensing assembly according to the present invention.

FIG. 2 is an expanded cross-sectional view of a glucose-sensing element of the glucose sensing assembly of FIG. 1.

FIG. 3 is a top perspective of the glucose sensing assembly of FIG. 1.

FIG. 4 is a bottom perspective of the glucose sensing assembly of FIG. 1.

FIG. 5 is a side view of the glucose sensing assembly of FIG. 1.

FIG. 6 is a set of graphs showing current versus time for a glucose-sensing element, such as that of FIG. 2, when introduced into any one of three different concentrations of glucose.

FIG. 7A is a perspective side view of a single use analyte sensor, in its undeployed state, according to an alternative embodiment of the present invention.

FIG. 7B is a perspective side view of the single use analyte sensor of FIG. 7A, in its deployed state.

FIG. 7C is a perspective view of a variant body fluid trap, that could be used as part of the embodiment of FIGS. 7A and 7B.

### DETAILED DESCRIPTION OF THE PREFERRED EMBODIMENT

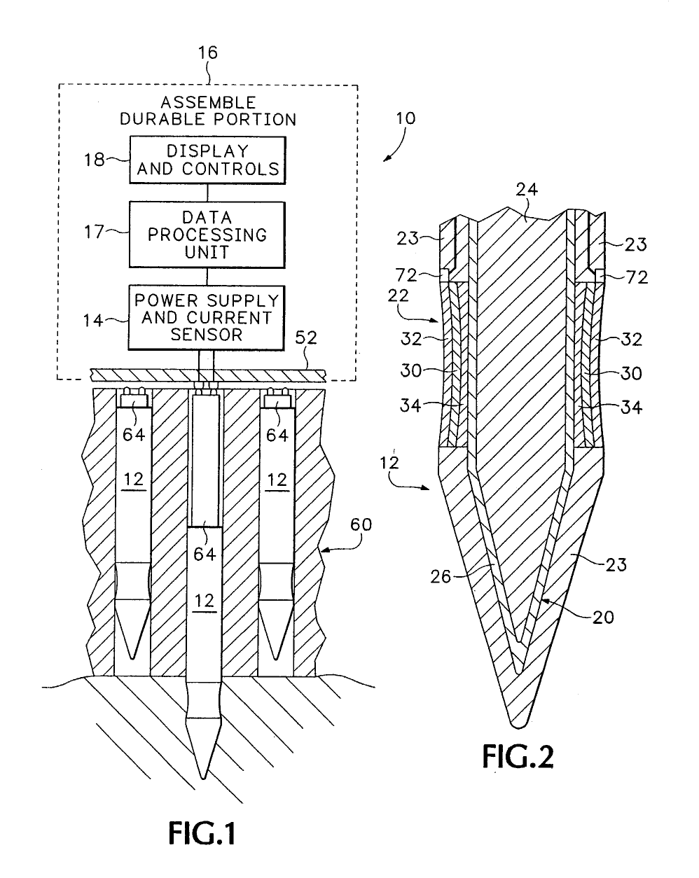

Referring to FIG. 1 a preferred embodiment of the present invention is a glucose sensor assembly 10 having a sensing element 12 that is adapted to be briefly introduced into the soft tissue of a patient. A power supply and current sensor unit 14, which is housed in an assembly durable portion 16, supplies the sensing element 12 with power. Sensing unit 14 quickly (<20 seconds) responds to introduction into the soft tissue of a patient by producing a sensing current that is generally proportional to the glucose level in this tissue. This current may be considered a “raw analyte measurement.” A data processing unit 17, measures the magnitude of this current, computes a best estimate glucose concentration (or a “refined analyte measurement”) and sends this information to a controls and display unit 18, which displays this estimate on a small liquid crystal display. Units 14, 17 and 18 may be collectively termed an “electric power, data processing and display device.” It should be noted that although in one preferred embodiment, described above, a fixed voltage is provided and a current is measured, in an alternative preferred embodiment, a fixed current is provided and the voltage across sensing element 14 is measured and is accordingly, the “raw analyte measurement.”

Because sensing element 12 yields the sensing current during its brief indwell period, there is no need to withdraw body fluid from the body. As a result, sensing element 12 is thinner at its distal end (<300 micrometers thick) than lancets adapted to create a puncture through the skin that is sufficient to permit body fluid flow therethrough.

Referring to FIG. 2, sensing element 12 includes a bimetallic needle 20 that in conjunction with a membrane system 22 reacts to the presence of glucose and oxygen to act as an indicating electrode. Needle 20 is coated with a protective layer 23, made of durable, non-toxic material such as polyimide, except for where coated by membrane system 22. In production, protective layer 23 is dip-coated onto needle 22 and then removed, preferably with an excimer or ND:YAG laser, in the area in which membrane system 22 is to be applied.

Needle 20 has a diameter of 227 microns and has a needle core 24 of structurally robust material such as stainless steel that is 226 microns thick and an electrochemically active plating 26, such as platinum, that is less than a micron thick.

The membrane system 22 must perform a number of functions. First, it must provide an enzyme that reacts with glucose and oxygen to form an electrolyte. A reactive layer 30 of glucose oxidase, glutaraldehyde and albumin produces hydrogen peroxide when contacted by glucose and oxygen, performs this function.

Second, because glucose is far more prevalent in the body fluid and other body fluids than oxygen, system 22 must include a membrane placed over the reactive layer 30 to permit a greater permeation of oxygen than glucose, so that the glucose concentration measurement is not limited by the oxygen concentration in the immediately surrounding tissue. This function is performed by a permselective hard block/soft block copolymer layer 32. This layer is of the type described in U.S. Pat. Nos. 5,428,123; 5,589,563 and 5,756,632, which are hereby incorporated by reference as if fully set forth herein. Layer 32 is preferably less than 10 microns thick, to permit rapid permeation by glucose and oxygen.

Third, membrane system 22 must prevent interferents, such as acetaminophen, from corrupting the measurement by causing current flow unrelated to the presence of glucose. This function is performed by an inner interferent reducing layer 34 of a compound such as sulfonated polyether sulfone, polyamino-phenol, or polypyrrole, in one embodiment 3-amino-phenol, which quickly permits the permeation of the hydrogen peroxide, which causes the current flow indicative of the concentration of glucose. Persons skilled in the relevant arts will readily recognize that quick permeation is highly desirable in a briefly indwelling sensor so that a measurement may be quickly obtained.

To produce sensing element 12, first the interferent reducing layer 34 of 3-amino-phenol is solution-coated or electro polymerized onto the surface of platinum plating 26. Layer 34 may be from a few nanometers to 2 microns thick, to permit rapid permeation by H₂O₂ ions, thereby reacting very quickly to glucose concentration. Over this the reactive layer 30 of glucose oxidase is dip-coated or electrodeposited. Glutaraldehyde is deposited on the glucose oxidase to immobilize the glucose oxidase. The sensor is dip coated in the soft block/hard block copolymer 32. In the finished product, the surface of the sensing region 22 is slightly depressed relative the remainder of the surface of sensing element 12. In one embodiment, the glucose oxidase 30 is applied before layer 34, which is electrodeposited through layer 30.

For sensing assembly 10, in one embodiment, a case 52 of the durable portion 16 serves as a reference electrode. The user grasps the case 52 in his right hand (left hand if the patient is left handed) and pushes a sensing element 12 into either his left arm or a left hand fingertip. When sensing element 12 enters the user's flesh, the circuit is completed through the user's body. Other reference electrode structures can consist of the portion of the housing immediately surrounding the indicating electrode or a patch placed upon the skin.

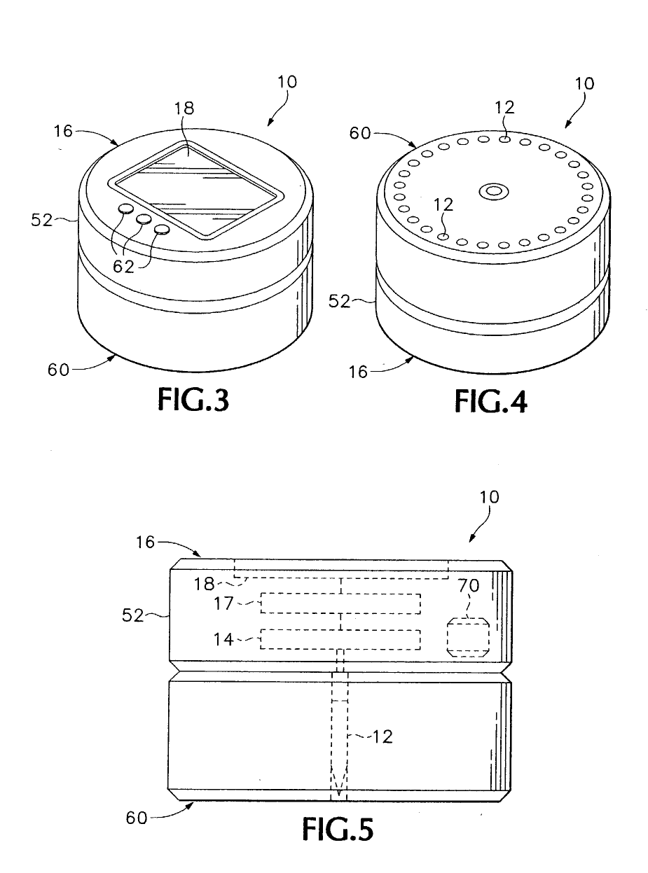

Referring to FIGS. 3-5, sensing elements 12 are preferably disposable and are provided in a disposable magazine 60, which is releasably and matingly attached to durable portion 16. Magazine 60 is rotated either manually or by pressing one of a set of buttons 62 that are part of the controls and display unit 18. In the second option a small electric motor 70 is actuated that rotates magazine 60. In either embodiment, an unused sensing element is rotated to an activation position, where it is aligned with an opening through the bottom of case 52. When a sensing element 12 is rotated into place, it is automatically electrically connected to the power supply and current sensing unit 14. On command from one of the buttons 62, or in an alternative embodiment always upon arriving sensing element 12 arriving in place, a sensor physical actuation unit 64 pushes sensing element 12 outwardly so that it will enter the flesh of the patient if assembly 10 is correctly positioned against the patient's skin.

In one preferred embodiment the sensing element 12 is energized to a voltage level of 0.65 volts between cathode and anode contemporaneously with being introduced into the patient. In one embodiment a pair of contacts 72 positioned at the top of the sensing region 22 are electrically connected by body fluid when the sensing region 22 is entirely covered by body fluid. This is used to trigger the application of voltage to the sensing element 12.

After insertion the patient waits for approximately six seconds at which time the controls and display unit 18 provides a reading of the body fluid glucose level. After this, unit 64 pulls sensing element 12 back into magazine 60, where it is stored until all of magazine 60 is detached from the durable portion 16 and placed in a proper disposal receptacle. Subsequently, a new magazine 60 is attached into durable portion 16, for further measurements. To support this option, in a preferred method, new magazines 60, filled with sensors 12 are made and sold. Alternatively, the entire assembly 10 is disposable and comes as a single non-separable unit.

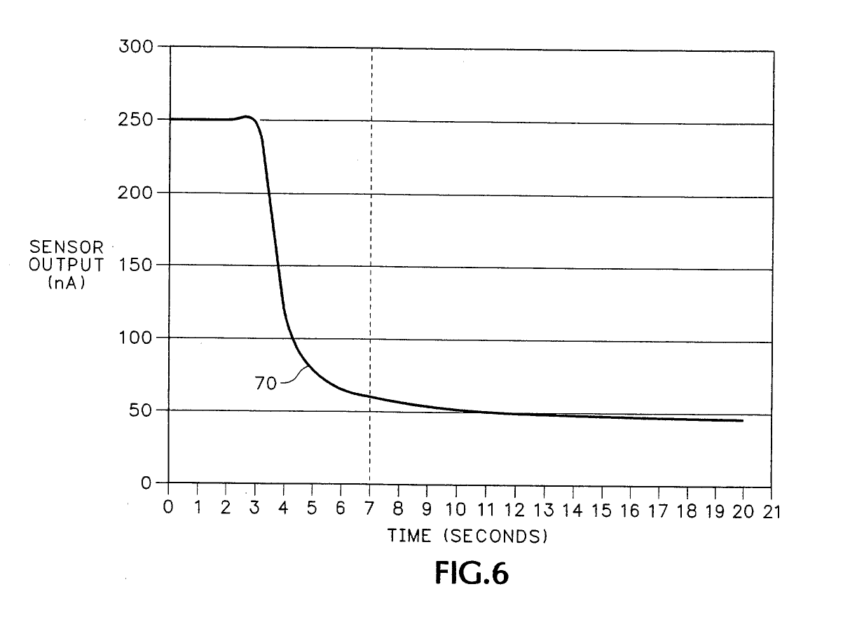

Referring to FIG. 6, which shows a graph 70 of early experimental readings (in current v. time coordinates) from a sensor according to the present invention after being placed in a solution of 5 mM glucose. The current reading at first entry into the solution is heavily corrupted by platinum oxidation and other, incompletely understood, factors. In one preferred embodiment the data processing unit 17, corrects the sensing element current by subtracting away a quantity representing the initial transient current at the moment the sensing element current is measured. To do this, a timing process is started at the moment when sensing element current is first detected. In one embodiment a set of corrected current measurements are averaged together to provide the reading. The correction for the anticipated initial transient current and the use of ultra-thin (for some embodiment less than 10 nanometers thick) membranes permits the calculation of a glucose concentration measurement within 20 seconds of the sensor being introduced into the patient's flesh. As noted, in a preferred embodiment a reading is provided in about 5 seconds. It is possible, in fact likely, that the glucose level will be changing as the unit is in the body.

Accordingly, in one preferred embodiment the direction of change, after correction for the initial transient current, is computed and displayed. In another embodiment a weighted average is formed of the corrected measurements, with more recent measurements being weighted more heavily, to give the patient a reading that reflects more heavily the more recent measurements. In yet another preferred embodiment, the processor simply waits until the initial transient current has dissipated, and then computes an instantaneous or brief period glucose concentration at the latest possible moment, in order to provide the patient with the timeliest information.

In an additional preferred embodiment, the signal processing unit 17 time tags and stores each glucose concentration estimate and uses this stream of time tagged estimates to send a value representing glucose concentration change to the controls and display unit 18.

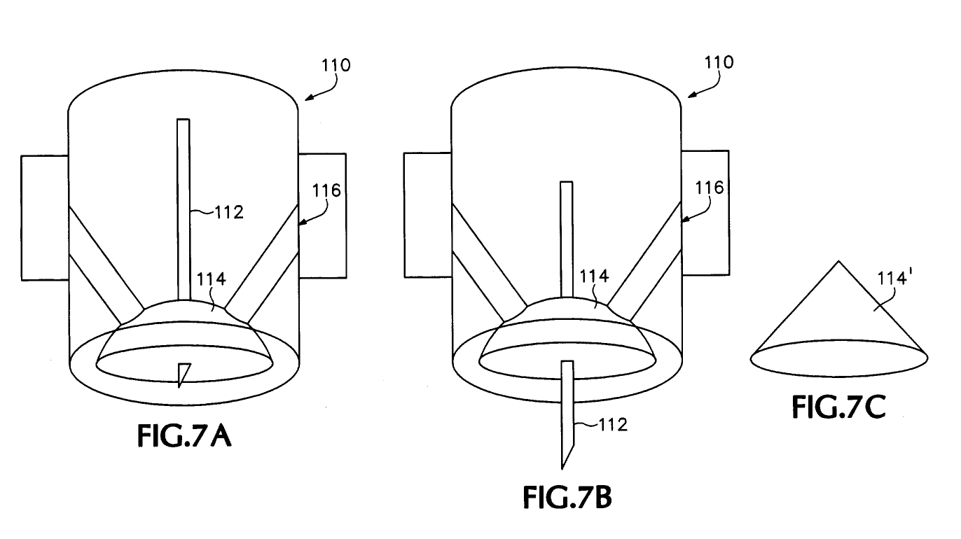

Referring to FIGS. 7A-7C, in an additional preferred embodiment of an analyte sensor assembly 110, a lancet 112 is provided to puncture the skin and a body fluid trap 114 is provided to retain the body fluid drawn from the broach created by lancet 112. Lancet 112 is somewhat thicker (>300 micrometers thick) than sensing element 12, to ensure that body fluid is drawn from the wound created. A pair of air vents 116 is provided to permit air to vacate trap 114. In this embodiment lancet 112 is quickly withdrawn, thereby avoiding pain to the patient. In one preferred embodiment lancet 112 is also the indicating electrode and includes a membrane system, such as 22 (FIG. 2), to create an analyte related current, with trap 114 serving as the reference electrode. In an alternative preferred embodiment, trap 114 is the indicating electrode and is coated with a membrane system such as system 22. In yet another preferred embodiment, lancet 112 serves as the indicating electrode. As the trapped body fluid touches the skin, the electrical current may easily flow through the trapped body fluid and through the patients skin, to the patients body, which will be touching the case of the analyte sensing assembly, which may serve as a reference electrode. In one preferred embodiment the body fluid that is trapped is blood.

Trap 114 preferably defines only a small volume and quickly fills with body fluid after lancet 112 punctures the skin, permitting analysis and answer in only 5 to 10 seconds. In one preferred embodiment, trap 114 defines about 30 nanoliters of interior volume. In other preferred embodiments, the trap defines a volume of from 30 nanoliters to 100 nanoliters. In any event the trap volume is small enough so that it will fill with body fluid in an amount of time on the order of one second, after the skin is broached. Assembly 110, may alternatively include a differently shaped body fluid trap 114′, as shown in FIG. 7C.

The terms and expressions which have been employed in the foregoing specification are used as terms of description and not of limitation, and there is no intention, in the use of such terms and expressions, of excluding equivalents of the features shown and described or portions thereof, it being recognized that the scope of the invention is defined and limited only by the claims which follow.

1. A rapid response, single use analyte sensor, adapted to measure an analyte concentration in body fluid of a patient having skin having an outer surface, said sensor comprising:
(a) a lancet;
(b) a body fluid trapping structure, adapted to trap body fluid on said skin outer surface;
(c) an electrochemical analyte measurement system adapted to measure an analyte concentration in body fluid that is trapped on said surface of said skin, by said body fluid trapping structure.

2. The analyte sensor of claim 1, wherein said body fluid trapping structure defines an interior volume of less than 100 nanoliters.

3. The analyte sensor of claim 1, wherein said body fluid trapping structure defines an interior volume of about 30 nanoliters.

4. The analyte sensor of claim 1, wherein said body fluid trapping member is more specifically adapted to trap blood.

5. A method of measuring the concentration of an analyte in the body fluid of a patient having skin having an outer surface, said method comprising:
(a) providing a rapid response, single use analyte sensor, adapted to measure an analyte concentration, said sensor comprising:
(i) a lancet;
(ii) a body fluid trapping structure, adapted to trap body fluid on said skin outer surface;
(iii) an electrochemical analyte measurement system adapted to measure an analyte concentration in body fluid that is trapped on said surface of said skin, by said body fluid trapping structure
(b) puncturing said skin with said lancet and withdrawing said lancet from said skin, thereby permitting body fluid to flow into said body fluid trapping structure;
(c) using said electrochemical analyte measurement system to measure said analyte concentration in said body fluid.

6. The method of claim 5, wherein said body fluid trapping structure defines an interior volume of less than 100 nanoliters.

7. The method of claim 5, wherein said body fluid trapping structure defines an interior volume of about 30 nanoliters.

8. The method of claim 5, wherein said body fluid trapping structure is blood.
